(19) United States
(12) Patent Application Publication
(10) Pub. No.: US 2023/0173498 A1
(43) Pub. Date: Jun. 8, 2023

(54) Partition Panel

(71) Applicant: BINDER GmbH, Tuttlingen (DE)

(72) Inventors: Gerhard STRAUB, Donaueschingen (DE); Ahmet ARABUL, Tuttlingen (DE); Richard LEIDOLT, Singen (DE)

(73) Assignee: BINDER GmbH, Tuttlingen (DE)

(21) Appl. No.: 18/073,800

(22) Filed: Dec. 2, 2022

(30) Foreign Application Priority Data
Dec. 3, 2021 (DE) 10 2021 131 981.1

(51) Int. Cl. B01L 9/00; B01L 1/00

(52) U.S. Cl. CPC B01L 9/00; B01L 1/00; B01L 2300/0858

(57) ABSTRACT

The disclosure relates to a partition panel for the detachable insertion into a laboratory chamber, for example an incubator, cold chamber, heating chamber, drying chamber of climate chamber, wherein the partition panel comprises a substantially rectangular flat base body with a lower edge, an upper edge, a front edge, a back edge as well as two parallel side faces, wherein on the base body at least one insertion hook open toward the front edge and at least one spring element for generating a force in the direction toward the front edge are disposed.

### CROSS REFERENCE TO RELATED APPLICATIONS

This application claims priority to German Patent Application No. 10 2021 131 981.1, filed Dec. 3, 2021, the entirety of which is incorporated by reference.

### FIELD OF APPLICATION

The application relates to a partition panel to be detachably inserted in a laboratory chamber, for example an incubator, cold chamber, heating chamber, drying chamber or climate chamber.

### BACKGROUND

Known are laboratory chambers with a housing comprising at least one outer door, which comprises at least one interior volume. Such laboratory chambers often comprise devices with which in the interior volume a defined temperature and/or defined climate conditions, for example a defined humidity or gas concentration, are settable. For example, incubators, cold chambers, heating chambers, drying chambers or climate chambers are known. It is known to close the interior volume using an additional inner door to prevent disturbing the climatic conditions in the interior volume when opening the outer door. The outer door serves substantially for insulation between the interior volume of the laboratory chamber and the work environment. The inner door prevents the air exchange between the interior volume and the work environment when opening the outer door. The inner door can be fabricated of a transparent material which, with the outer door open, allows observing the interior volume without disturbing the climatic conditions in the interior volume. In this case there is the feasibility of dividing the inner door into several smaller doors which only permit access into one subregion of the interior volume. An incubator with such an inner door is shown in DE 10 2004 049 210 A1 for example. A compartment herein extends over the entire width of the laboratory chamber.

US 2005/0244306 A1 discloses a climate chamber in which the interior volume is divided by boxes into several subvolumes and several inner doors close off the smaller subvolumes such that the climate in the remaining subvolumes is as little disturbed as is feasible when into one of the subvolumes samples are being placed for storage or samples are removed from the subvolume. Such implementation lacks flexibility and is highly complex.

### SUMMARY

The application addresses the problem of providing a feasibility for being able to flexibly subdivide the interior volume of a laboratory chamber into smaller regions in simple manner.

According to the disclosure the problem is resolved by a partition panel to be detachably inserted into a laboratory chamber.

Advantageous embodiments and further developments of the disclosure are specified in the dependent claims.

The partition panel for the detachable insertion into a laboratory chamber, for example into an incubator, cold chamber, heating chamber, drying chamber or climate chamber, comprises a substantially rectangular, flat base body with a lower edge, an upper edge, a front edge, a back edge as well as two parallel lateral faces, wherein on the base body at least one insertion hook open toward the front edge and at least one spring element for generating a force in the direction toward the front edge are disposed. Such implementation enables simple inserting and detaching of the partition panel without additional tools since through the spring element fixation can be completed which can also be released again in simple manner. Such a partition panel enables flexible compartmentalization: depending on the requirement, the partition panel can be inserted in order to obtain smaller subregions or it can be removed should larger subregions be required.

The partition panel according to the disclosure can be disposed on an, in particular, horizontally disposed bottom or intermediate tray of a laboratory chamber such that it is oriented perpendicularly to the bottom or the intermediate tray and thus is, in particular, oriented vertically. In addition to a basically known division into compartments which is obtained through the intermediate trays, with the partition panel according to the disclosure a further division on a compartment level into smaller compartments can be achieved in order to decrease therewith the risk of interchanging samples, especially when several doors are disposed adjacent to one another on one compartment level.

In one embodiment of the disclosure, at least two insertion hooks are disposed on the base body whereby the fixation stability can be increased.

In one embodiment of the disclosure, the insertion hooks are disposed on the lower edge which enables an especially space-saving disposition.

Further development of the disclosure the spring element is formed by a canted strap, in particular a double-canted strap. Such spring element can be simply produced by bending.

The spring element is advantageously implemented to be approximately V-shaped whereby a space-saving physical form of the spring element results. Such spring element, moreover, can be especially simple to manipulate.

In one embodiment of the disclosure, a free end of the spring element is developed as a seating element which can simplify manipulating the spring element when inserting the partition panel.

The disclosure provides that at the lower edge at least one support element is disposed, which is implemented substantially perpendicular to a side face, wherein in one embodiment of the disclosure at least two support elements pointing, in particular, in different directions, are disposed on the lower edge. Such support element can increase the stability of the partition panel and decrease the risk of the partition panel from tilting in the direction in which the support element points.

In one embodiment of the disclosure, the spring element is disposed on the lower edge, a space-saving physical form can result. In an alternative, in another embodiment the spring element can be disposed on the rear edge in spatial proximity to the lower edge or on the support element, which can enable a single-piece implementation between spring element and base body and, in particular, fabrication using a stamping-bending process.

In one embodiment, the base body comprises a multiplicity of perforations and is, in particular, developed as a perforated metal plate. Such physical form of the partition panel can prevent the partition panel from impairing the spatial temperature or humidity distribution in the interior volume of the laboratory chamber.

The partition panel is advantageously fabricated of a metal. A partition panel of metal enables simple cleaning and, in particular, sterilization.

According the disclosure the partition panel is developed as a stamped-and-bent element and in particular as a single-piece element. Such partition panel permits the especially simple and cost-effective production.

A laboratory chamber according to the disclosure, for example an incubator, cold chamber, heating chamber, drying chamber or climate chamber with a housing comprising at least one outer door, which housing comprises at least one interior volume that can be closed off by an inner door, wherein the interior volume comprises a bottom wall, a top wall, a back wall and two side walls, wherein between the two side walls parallel to the bottom wall at least one intermediate tray is disposed, wherein either the bottom wall and/or the intermediate tray comprises or comprise a first cutout and an abutment edge, comprises a partition panel according to the disclosure which is disposed on the bottom wall and/or on the intermediate tray such that the insertion hook engages into the cutout and the spring element is in contact on the abutment edge.

### BRIEF DESCRIPTION OF DRAWINGS

Embodiments of the application will be described in detail in conjunction with the following Figures. Therein depict:

FIG. 1 a perspective view onto a first embodiment example of a disposition of components disposed in a housing of a laboratory chamber, in particular comprising two intermediate trays as well as two partition panels disposed on the intermediate trays, wherein the housing is hidden,

FIG. 2 a view from above onto the disposition according to FIG. 1,

FIG. 3 a cut-out enlargement of detail Z of FIG. 2 showing a spring element,

FIG. 4 a view from the front onto the disposition according to FIG. 1,

FIG. 5 a view from obliquely below onto the disposition according to FIG. 1,

FIG. 6 a cut-out enlargement of detail Y of FIG. 5 which shows an insertion hook,

FIG. 7 a cut-out enlargement of a further perspective view of the disposition according to FIG. 1,

FIG. 8 a perspective view onto a second embodiment example of a disposition of components disposed in a housing of a laboratory chamber, in particular comprising two intermediate trays as well as two partition panels disposed on the intermediate trays, wherein the housing is hidden,

FIG. 9 a view from above onto the disposition according to FIG. 8,

FIG. 10 a view from the front onto the disposition according to FIG. 8,

FIG. 11 a view from obliquely below onto the disposition according to FIG. 8,

FIG. 12 a partially sectioned further perspective view onto the disposition according to FIG. 8,

FIG. 13 a cut-out enlargement of detail A of FIG. 11 showing an insertion hook,

FIG. 14 a cut-out enlargement of detail B of FIG. 11 showing a spring element,

FIG. 15 the spring element according to FIG. 14 in a further perspective view,

FIG. 16 the spring element according to FIG. 14 in a further perspective view before insertion of the partition panel,

FIG. 17 the spring element according to FIG. 14 in a further perspective view after insertion of the partition panel,

FIG. 18 a side view of the partition panel of the disposition according to FIG. 8,

FIG. 19 a cut-out enlargement of a perspective view of the disposition according to FIG. 18,

FIG. 20 a perspective view of an embodiment example of a laboratory chamber with the components disposed therein,

FIG. 21 a further perspective view of the laboratory chamber according to FIG. 20 with partially pulled-out intermediate trays,

FIG. 22 a view from the front of the laboratory chamber according to FIG. 20,

FIG. 23 a sectional view of the laboratory chamber according to FIG. 20 and

FIG. 24 a further perspective view of the laboratory chamber according to FIG. 20.

### DETAILED DESCRIPTION

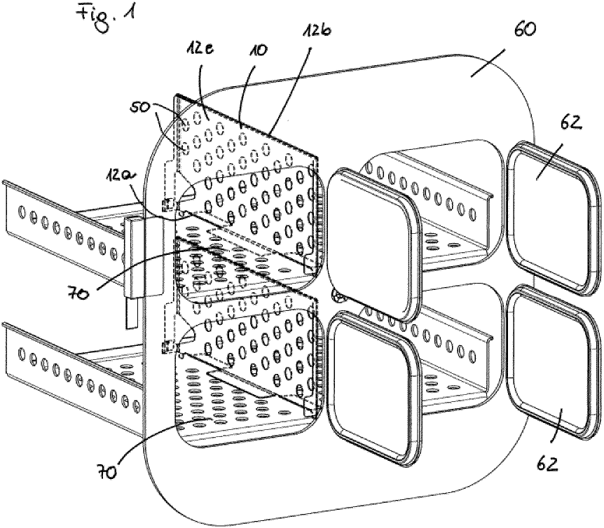

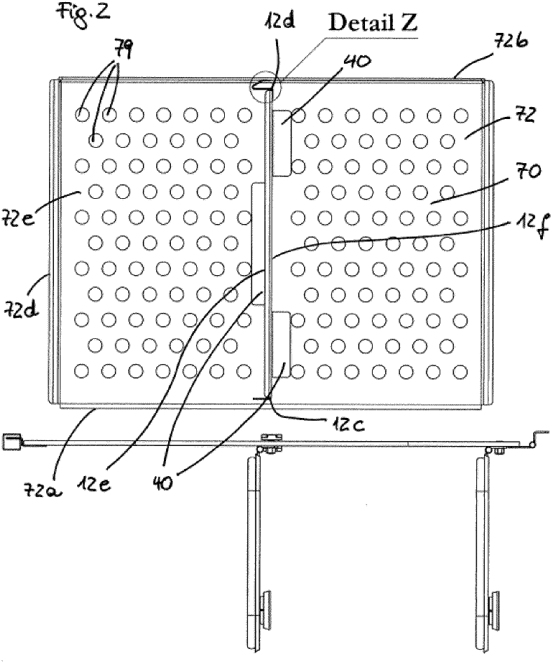

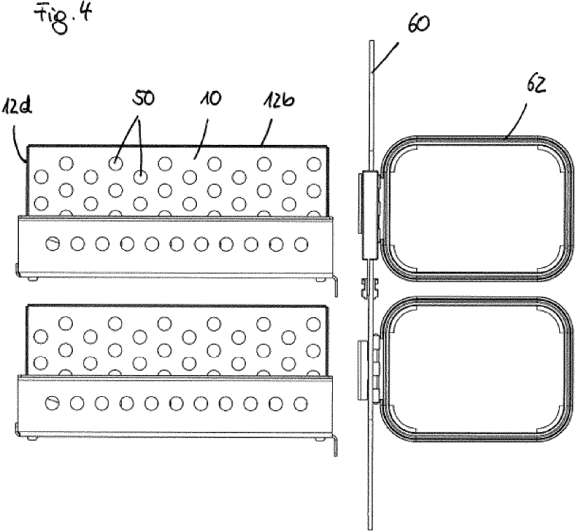

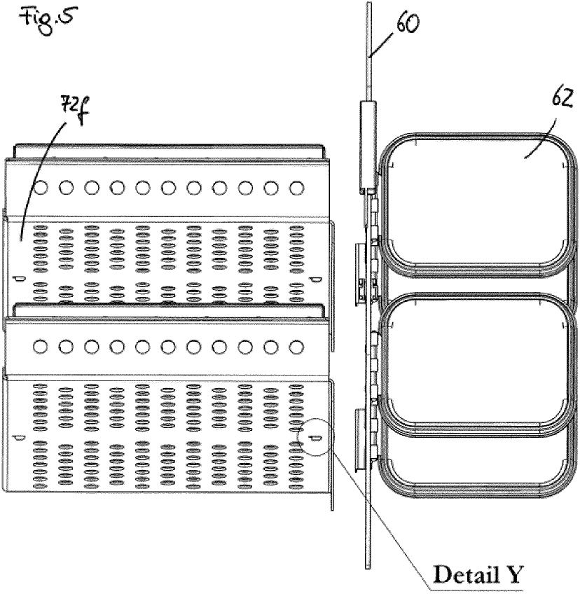

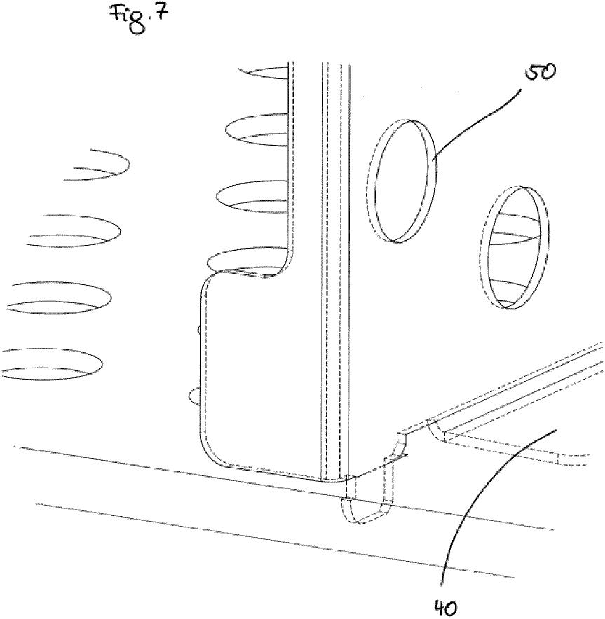

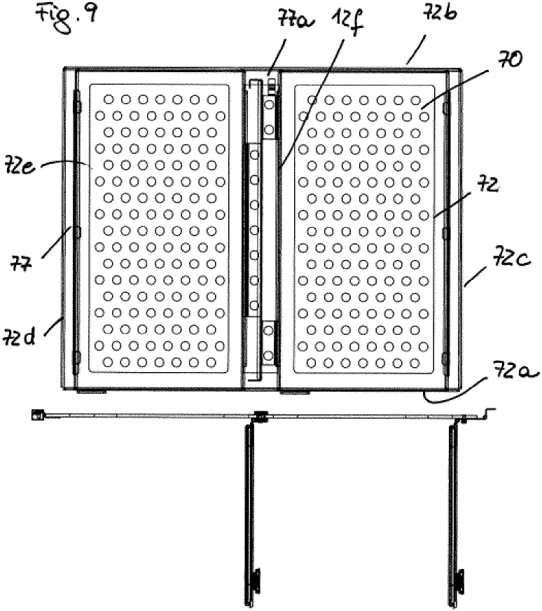

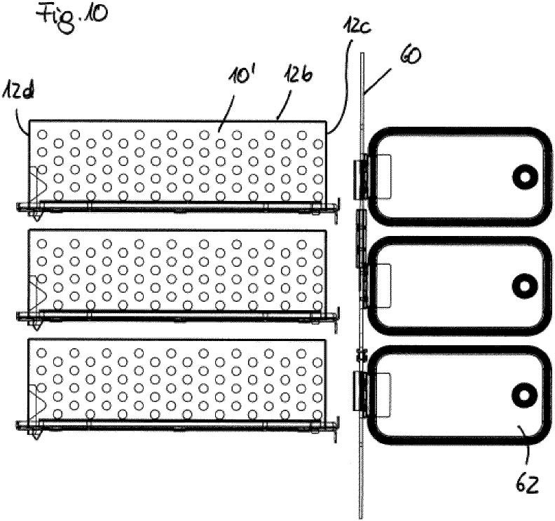

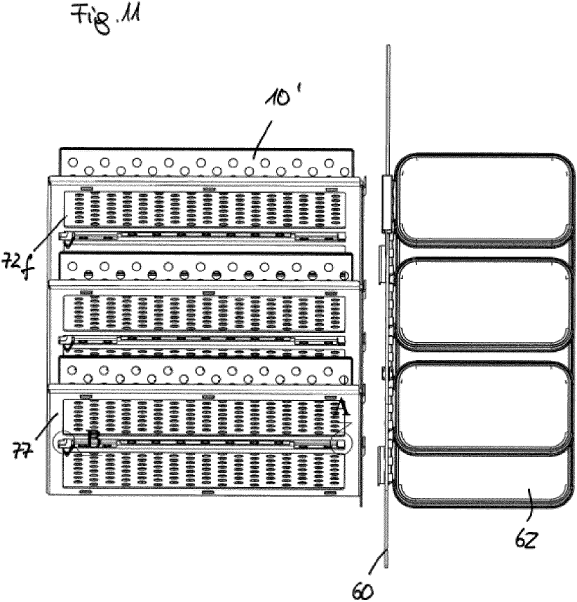

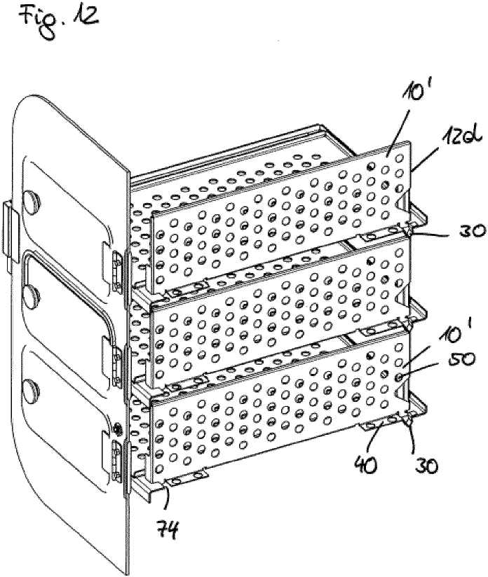

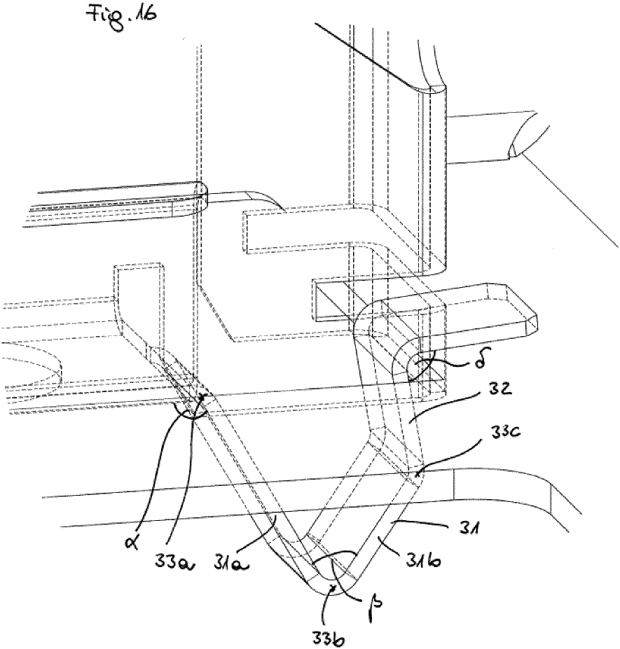

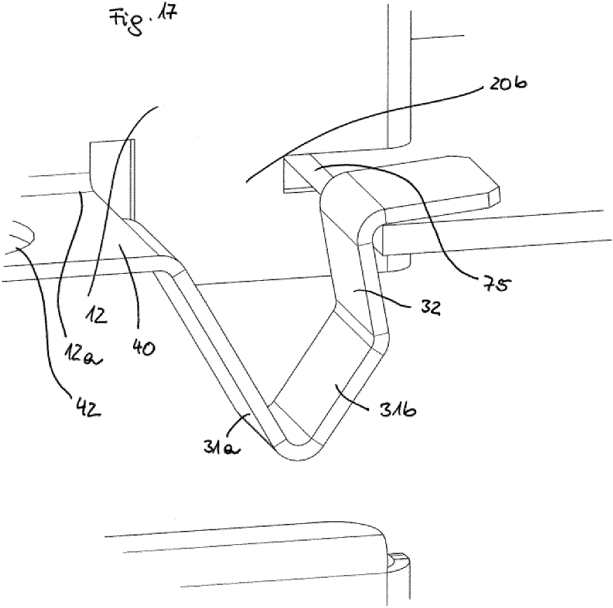

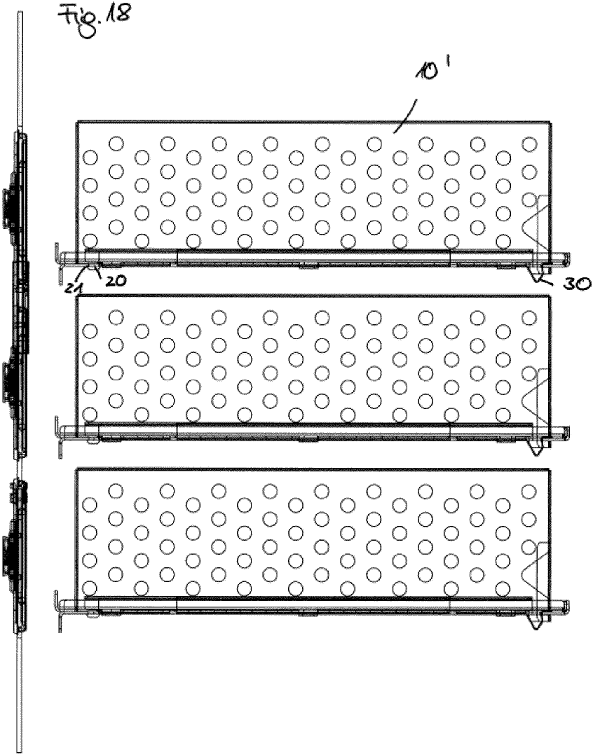

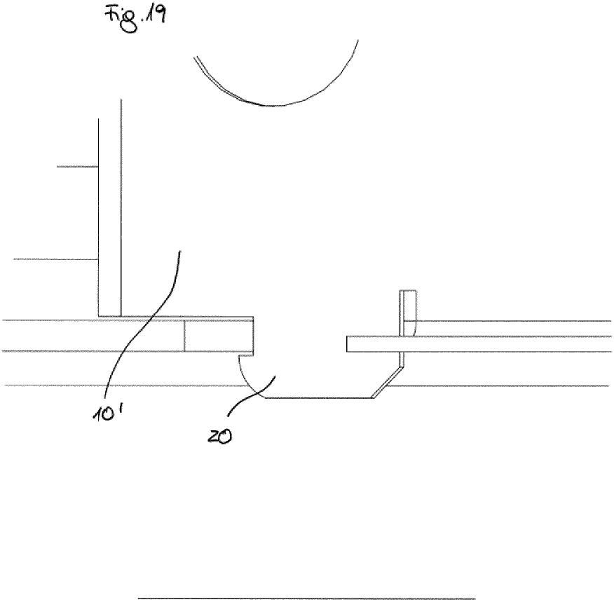

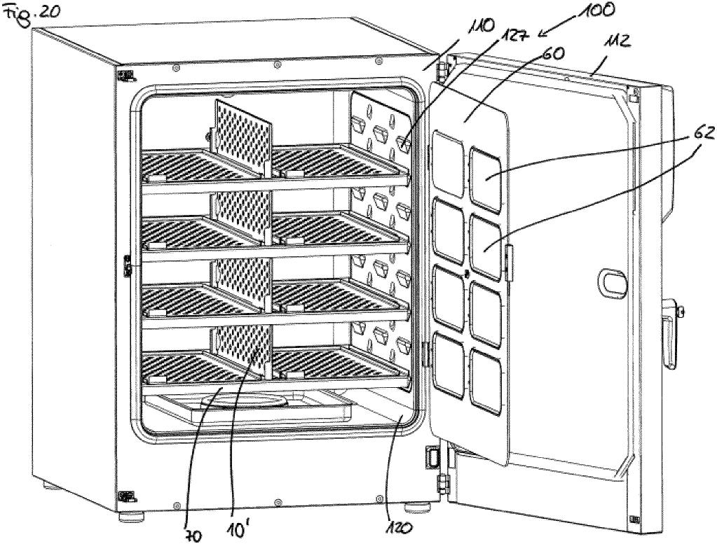

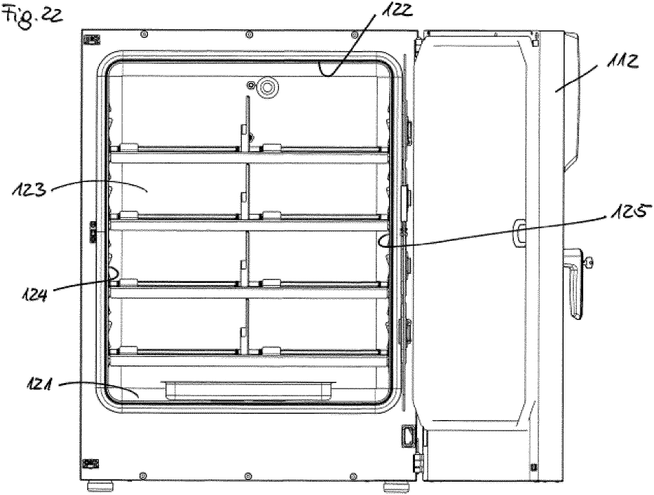

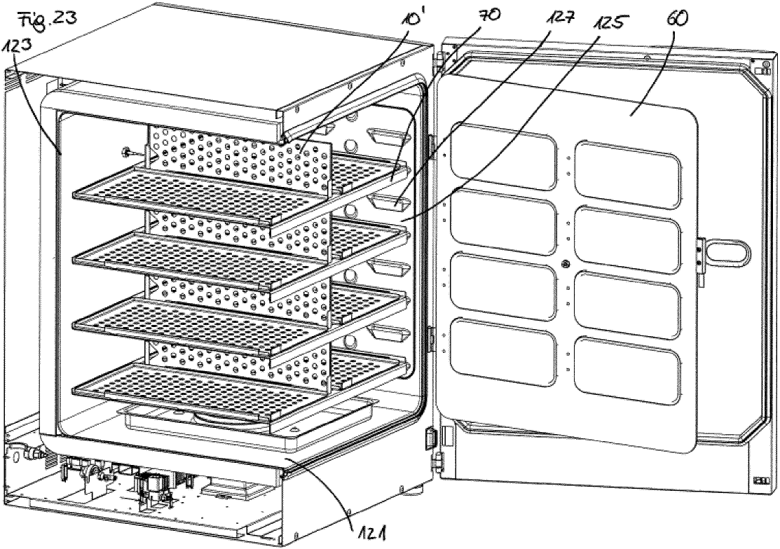

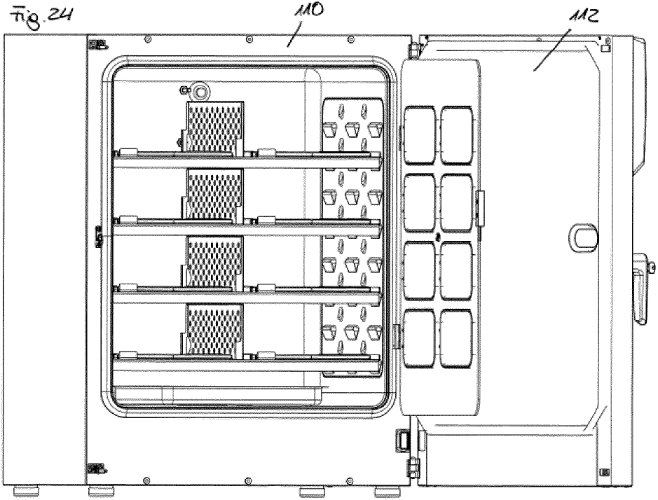

FIGS. 1 to 8 show different views of a first embodiment example of a disposition of components disposed in a housing of a laboratory chamber, wherein the housing is hidden while FIGS. 9 to 19 show different views of a second embodiment example of a disposition of components disposed in a housing of a laboratory chamber wherein the housing is hidden. FIGS. 20 to 24 show different views of an embodiment example of a laboratory chamber 100 in which the disposition of components according to the second embodiment example depicted in FIGS. 9 to 19 is disposed. Like or functionally like parts are denoted with like reference numbers, wherein, for the sake of a clearer view, not all reference numbers are shown in all Figures. Unless explicitly stated otherwise, the following description refers to both embodiment examples of the dispositions. In particular, both embodiment examples of the dispositions can be disposed in the laboratory chamber 100 according to FIGS. 20 to 24.

FIG. 1 shows inter alia a partition panel 10 for the detachable insertion into a laboratory chamber 100, for example an incubator, cold chamber, heating chamber, drying chamber or climate chamber, not depicted in this Figure. The partition panel 10 comprises a substantially rectangular flat base body 12 with a lower edge 12a, an upper edge 12b, a front edge 12c and back edge 12d as well as two parallel side faces 12e, 12f, wherein the lower edge 12a is disposed in particular parallel to the upper edge 12b and the front edge 12c is disposed, in particular, parallel to the back edge 12d. The distance between the two side faces 12e, 12f is herein in particular substantially less than the length of the front edge 12c and the length of the lower edge 12a such that a flat base body 12 results. The base body 12 can in particular comprise perforations 50 and be implemented, for example, as a perforated metal plate in order to impair as little as feasible the air circulation in the interior volume of the laboratory chamber 100.

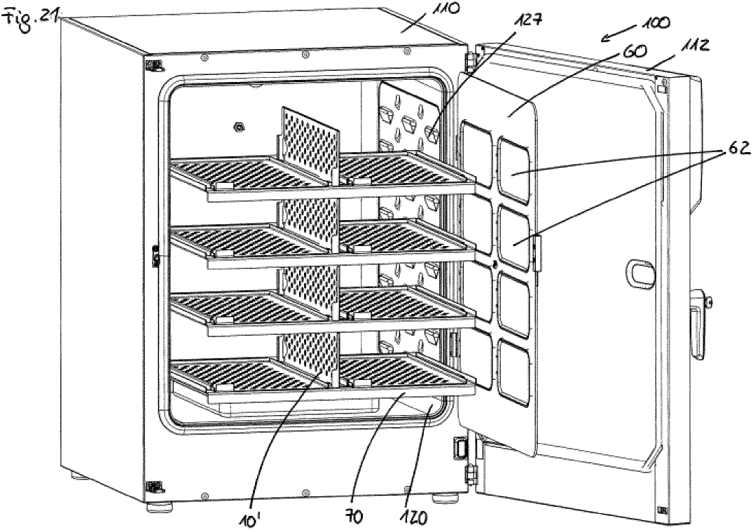

As is discernable in FIGS. 20 to 24, the laboratory chamber 100 conventionally comprises at least a housing 110 comprising an outer door 112, which housing 110 comprises at least one interior volume 120 that can be closed off by an inner door 60. The inner door 60 can herein comprise several small doors 62 each enabling only access to one subregion of the interior volume 120. The interior volume 120 herein comprises conventionally a bottom wall 121, a top wall 122, a back wall 123 and two side walls 124, 125. The side walls 124, 125 and the back wall 123 are herein conventionally substantially vertically disposed while the bottom wall 121 and the top wall 122 are conventionally horizontally disposed. Between the two side walls 124, 125 at least one intermediate tray 70 is disposed parallel to the bottom wall 121, wherein in the embodiment example depicted in FIG. 1 two intermediate trays 70 are depicted. The intermediate tray 70 comprises a substantially rectangular flat base body 72 with a front edge 72a, a back edge 72b and two parallel side edges 72c, 72d as well as an upper side 72e and a lower side 72f. The distance between the upper side 72e and the lower side 72f is herein in particular substantially less than the length of the front edge 72a and the length of the side edges 72c, 72d such that a flat base body 72 results. The base body 72 can in particular comprise perforations 79 and be developed, for example, as a perforated metal plate in order to impair the air circulation in the interior volume 120 of the housing 110 as little as feasible. The intermediate tray 70 can be slid into corresponding receiving tracks or beadings 127 which are disposed in the interior volume 120 of the housing 110 (cf. in particular FIG. 21).

When the partition panel 10 is used as intended when inserted in the interior volume 120 of the laboratory chamber 100 and, in particular, when disposed on the intermediate tray 70, the lower edge 12a points downward and the upper edge 12b points upward such that the partition panel 10 is substantially vertically disposed. Furthermore, when the partition panel 10 is used as intended the front edge 12c is disposed directed toward the inner door [60] while the back edge 12d is disposed directed toward the back wall [123] of the interior volume [120]. However, a disposition such that the front edge 12c is disposed directed toward the back wall [123] and the back edge 12d is directed toward the inner door [60] is also feasible.

On the base body 12 of partition panel 10 at least one insertion hook 20 open toward the front edge 12c, two insertion hooks 20 open toward the front edge 12c, is or are disposed, in particular on the lower edge 12a of partition panel [10]. The insertion hook 20 can comprise a feed opening 21 which is open toward the front edge 12c. The insertion hook 20 is in particular of the same thickness as the base body 12 and can thus be disposed in particular in extension of the base body 12 on the lower edge 12a. The insertion hook 20 can in particular be developed in the same production step as the stamping of the partition panel 10.

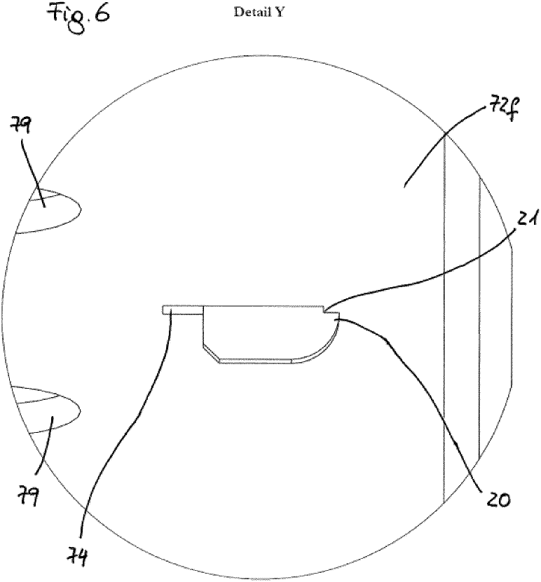

In the disposition of the partition panel 10 on the intermediate tray 70 the insertion hook 20 is set into a cutout 74 disposed in the intermediate tray 70 in particular such that the margin of the cutout 74 engages into the feed opening 21 of the insertion hook 20. The insertion hook 20 herein passes through the cutout 74 such that the base body 20 with its lower edge 12a is in contact on the upper side 72e of the intermediate tray 70 while the free end of the insertion hook is in contact on the lower side 72f of the intermediate tray 70 (cf. FIG. 6). The partition panel 10 can also be disposed on the bottom of the interior volume 120.

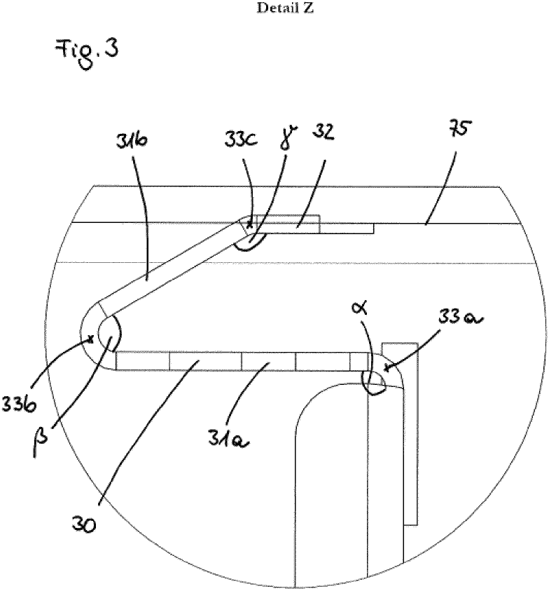

On the base body 12 of the partition wall 10 at least one spring element 30 is disposed for the generation of a force in the direction toward the front edge 12c (cf. in particular FIG. 3). Such spring element 30 effects in particular that the edge of cutout 74 is pressed against the bed of the insertion hook 20 such that reliable fixation of the partition panel 10 can be achieved.

The spring element 30 can be implemented, for example, as a canted strap 31, in particular by a double-canted strap 31. The strap 31 can herein be canted at a first bending edge 33a with respect to the base body 12 such that a first section 31a of the strap 31 is disposed at an inclination by an angle a with respect to the base body 12. The bending edge 33a [can] extend for example parallel to the back edge 12d. The angle a can be, for example approximately 90°. The strap 31 is doubly canted. To this end, the strap 31 can be canted at a second bending edge 33b, which extends, in particular, parallel offset with respect to the first bending edge 33a such that a second section 31b results which is disposed at an inclination by an angle β with respect to the first section 31a. The angle β is less than 90° and be, for example, between 20° and 60°, preferably between 30° and 40°. The spring element 30 is, in particular, developed to be approximately V-shaped.

A free end of the spring element 30 can be developed as a seating element 32 and for this purpose be canted again. The seating element 32 can in particular be disposed at a third bending edge 33c disposed at an inclination with respect to the second section 31b by an angle γ.

In the present embodiment example, the spring element 30 is disposed on the back edge 12d in spatial proximity to lower edge 12a (cf. FIG. 1). Such disposition enables a single-piece disposition between the spring element 30 and the base body 12 as well as the implementation of the partition panel 10 as a stamped-and-bent element.

In the disposition of the partition panel 10 on the intermediate tray 70 the seating element 32 is in contact on an abutment edge 75. The spring element 30 is developed such that, after the engagement of the insertion hook 20 into the cutout 74 of the intermediate tray [70] and contact of the seating element 32 on the abutment edge 75, a force is generated which presses the edge of the cutout 74 into the bed of the insertion hook 20.

On the lower edge 12a of base body 12 at least one support element 40, which is substantially developed perpendicularly to one of the side faces 12e, 12f, can be disposed wherein at least two, in the present embodiment example three, support elements 40 are disposed on the lower edge 12a. Of the three support elements 40 the middle one points into the direction opposite to the two outer ones in order to be able to enable the highest feasible position stability of the partition panel 10.

The partition panel 10 is in particular fabricated of a metal. The partition panel 10 is especially developed as a single piece. The partition panel 10 is in particular developed as a stamped-and-bent element.

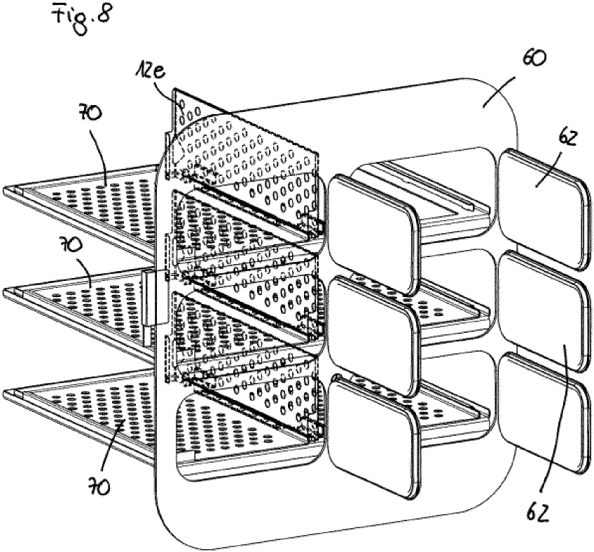

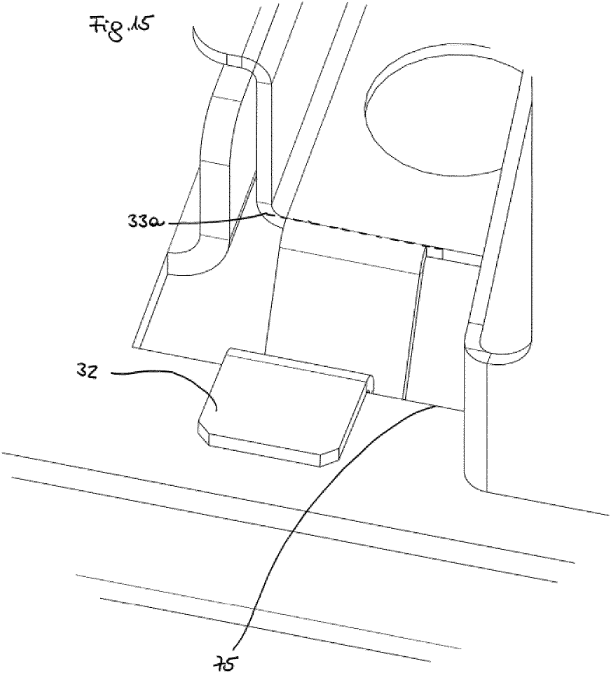

The second embodiment example depicted in FIGS. 8 to 19 differs essentially by the disposition of the spring element 30 on the base body 12 of partition panel 10′. Instead of on the back edge 12d, the spring element 30 can be disposed on the support element 40 (cf. in particular FIG. 15). The first bending edge 33a can herein be disposed in particular perpendicularly to the plane of the side faces 12e, 12f. In this embodiment example the seating element 32 is developed canted onto itself at an angle δ in order to be able to rest with the free end of the strap 31 on the upper side 72e of the intermediate tray 70. The angle δ can be, for example, between 80° and 90°.

In this embodiment example the intermediate tray 70 comprises a frame-like element 77 with a central web 77a wherein in the central web 77a the cutout 74 and the abutment edge 75 are disposed such that the partition panel 10′ can be disposed on the central web 77a. Laterally on each side of the central web 77a a perforated metal plate can be emplaced into the frame-like 77.

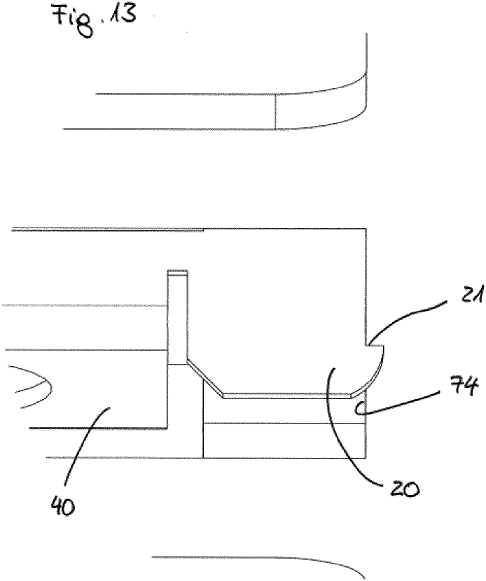

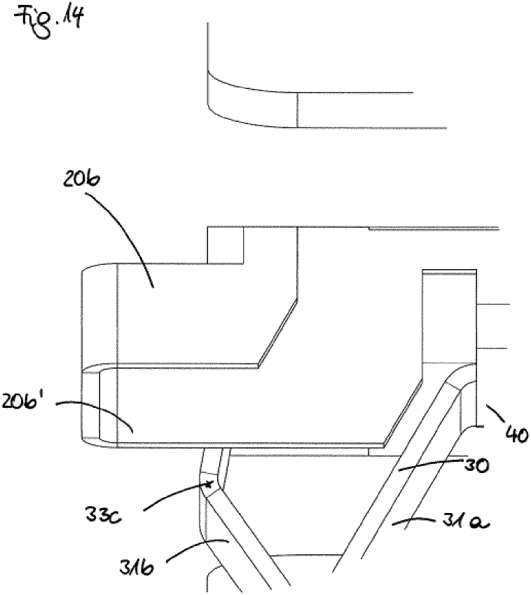

The second embodiment example of the partition panel 10′, in addition, differs from the first embodiment example of partition panel 10 thereby that only the insertion hook 20 disposed in spatial proximity to the front edge 12c is developed to be open toward the front edge 12c (cf. FIG. 13) while an insertion hook 20b disposed in spatial proximity to the back edge 12d is developed to be open toward the back edge 12d (cf. FIG. 14). The rearward insertion hook 20b serves therewith for stabilization. However, no pressing takes place by the spring element 30 through the edge of the cutout 74 toward the bed of the insertion hook 20b.

Furthermore, due to a U-shaped bending-over of the back edge 12d, two insertion hooks 20b, 20b′ disposed parallel offset with respect to one another can be developed (cf. FIGS. 14 to 17).

Lastly, the support elements 40 can, as shown in the second embodiment example, also comprise perforations 42.

If the partition panel 10. 10′ according to the first as well as also according to the second embodiment example, is disposed on the intermediate tray 70, it separates the region above the intermediate tray 70 into two subregions. If in the inner door 60 at the level above the intermediate tray 70 two small doors 62 are disposed next to one another, one subregion is accessible through each of the two small doors 62. The partition panel 10, 10′ can be mounted simply without any additional tools thereby that the insertion hook 20 is guided through the cutout 74 and the spring element 30 is brought into contact on the abutment edge 75. Removal of the partition panel 10, 10′ is also feasible in simple manner without additional tools by pressing the spring element 30 together against the spring force and lifting the partition panel 10, 10′ from the intermediate tray 70.

List of References

| | |
|---|---|
| 10 | Partition panel |
| 10′ | Partition panel |
| 12 | Base body |
| 12 a | Lower edge |
| 12 b | Upper edge |
| 12 c | Front edge |
| 12 d | Back edge |
| 12 e | Side face |
| 12 f | Side face |
| 20 | Insertion hook |
| 20 b | Insertion hook |
| 20b′ | Insertion hook |
| 21 | Feed opening |
| 30 | Spring element |
| 31 | Strap |
| 31 a | First section |
| 31 b | Second section |
| 32 | Seating element |
| 33 a | First bending edge |
| 33 b | Second bending edge |
| 33 c | Third bending edge |
| 40 | Support element |
| 42 | Perforation |
| 50 | Perforation |
| 60 | Inner door |
| 62 | Small door |
| 70 | Intermediate tray |
| 72 | Base body |
| 72 a | Front edge |
| 72 b | Back edge |
| 72 c | Side edge |
| 72 d | Side edge |
| 72 e | Upper side |
| 72 f | Lower side |
| 74 | Cutout |
| 75 | Abutment edge |
| 77 | Frame-like element |
| 77 b | Central web |
| 79 | Perforation |
| 100 | Laboratory chamber |
| 110 | Housing |
| 112 | Outer door |
| 120 | Interior volume |
| 121 | Bottom wall |
| 122 | Top wall |
| 123 | Back wall |
| 124 | Side wall |
| 125 | Side wall |
| 127 | Beading |
| α | Angle |
| β | Angle |
| γ | Angle |
| δ | Angle |

1. A laboratory chamber component, comprising:
an insertable and detachable laboratory chamber partition panel, comprising:
a substantially rectangular flat base body with a lower edge, an upper edge, a front edge, a back edge, and two parallel side faces,
an insertion hook that is located on the base body and that opens toward the front edge, and
a spring located on the base body and that generates a force toward the front edge.

2. The laboratory chamber component as in claim 1, wherein at least two insertion hooks are disposed on the base body.

3. The laboratory chamber component as in claim 1, wherein the insertion hook is disposed on the lower edge.

4. The laboratory chamber component as in claim 1, wherein the spring is a canted strap.

5. The laboratory chamber component as in claim 1, wherein the spring is a double-canted strap.

6. The laboratory chamber component as in claim 1, wherein the spring is approximately V-shaped.

7. The laboratory chamber component as in claim 1, wherein a free end of the spring is a spring seat.

8. The laboratory chamber component as in claim 1, further comprising a support extension disposed on the lower edge and substantially perpendicular to at least one of the two parallel side faces.

9. The laboratory chamber component as in claim 1, further comprising at least two support extensions disposed on the lower edge and substantially perpendicular to at least one of the two parallel side faces, wherein the least two support extensions point in different directions from each other.

10. The laboratory chamber component as in claim 8, wherein the spring is disposed on the lower edge, on the back edge in spatial proximity to the lower edge or on the support extension.

11. The laboratory chamber component as in claim 1, wherein the base body comprises a plurality of perforations.

12. The laboratory chamber component as in claim 11, wherein the base body is a perforated metal plate.

13. The laboratory chamber component as in claim 1, wherein the partition panel is a fabricated metal.

14. The laboratory chamber component as in claim 1, wherein the partition panel is a stamped-and-bent element.

15. The laboratory chamber component as in claim 14, wherein the partition panel is a single piece.

16. A laboratory chamber, comprising:
a laboratory chamber housing;
a outer door and an inner door;
an interior volume that can be closed off by the inner door, wherein the interior volume comprises a bottom wall, a top wall, a back wall and two side walls;
an intermediate tray disposed between the two side walls and parallel to the bottom wall, wherein either the bottom wall and/or the intermediate tray comprise a first cut-out and an abutment edge;
a partition panel, the partition panel being an insertable and detachable laboratory chamber partition panel, comprising:
a substantially rectangular flat base body with a lower edge, an upper edge, a front edge, a back edge, and two parallel side faces,
an insertion hook that is located on the base body and that opens toward the front edge,
a spring located on the base body and that generates a force toward the front edge,
wherein the partition panel is disposed on the bottom wall and/or on the intermediate tray such that the insertion hook engages into the cut-out and the spring is in contact on the abutment edge.
